(19) United States
(12) Patent Application Publication
(10) Pub. No.: US 2007/0298057 A1
(43) Pub. Date: Dec. 27, 2007

(54) COMPOSITION AND METHOD FOR MODULATING ADDICTIVE BEHAVIORS

(76) Inventor: Edward Larry McCleary, Incline Village, NV (US)

Correspondence Address: PATTON BOGGS LLP, 1801 CALFORNIA STREET, SUITE 4900, DENVER, CO, 80202, US

(21) Appl. No.: 11/833,474

(22) Filed: Aug. 3, 2007

Related U.S. Application Data
(62) Division of application No. 11/111,542, filed on Apr. 21, 2005.
Application No. 11/111,542 is a continuation-in-part of application No. 10/986,924, filed on Nov. 12, 2004.

(51) Int. Cl. A61K 36/53; A23L 1/30; A61K 36/82; A61P 25/30; A61K 9/00; A23L 2/00

(52) U.S. Cl. 424/400; 424/729; 424/745; 426/590; 426/648

(57) ABSTRACT

Improved compositions and methods useful in the treatment and prevention of impulsive, compulsive, reward seeking, and other addictive behaviors, including applications involving the behavioral/cognitive process of assigning importance, maintaining attention, and assessing salience. The composition includes Vitex agnus-castus (Chastetree berry) extract in an effective amount for supporting and promoting a healthy lifestyle free from addictive behavior or for preventing or treating addictive behavior. Huperzine and/or 5-HTP (5-hydroxytryptophan) may also be included. The composition may also include ingredients to address nutritional deficiencies in the addict, and nutrients depleted during the withdrawal of any reinforcer that is the object of the addiction.

### RELATED APPLICATIONS

This application is a divisional application of U.S. patent application Ser. No. 11/111,542 filed Apr. 21, 2005, which is a continuation-in-part of U.S. patent application Ser. No. 10/986,924 filed Nov. 12, 2004.

### BACKGROUND OF THE INVENTION

1. Field of the Invention

This invention relates in general to nutraceuticals, including functional foods, dietary supplements, nutritional supplements, medical foods, botanical drugs, and drugs. In particular, it relates to nutraceuticals and methods that are useful for supporting and promoting a healthy lifestyle free from impulsive, compulsive, reward seeking, and/or addictive behavior, including weight control vs. impulsive eating or bingeing.

2. Statement of the Problem

In this disclosure, the term addictive behavior includes behavior commonly categorized under the term addiction, as well as impulsive, compulsive, craving, and reward seeking behavior and including behaviors involving the behavioral/cognitive process of assigning importance, maintaining attention, and assessing salience. Studies using positron emission tomography (PET) have documented reductions in striatal dopamine receptors in drug addicts. However, the role of dopamine in addiction, i.e., loss of control and compulsive drug intake, is poorly understood. Neuroimaging studies have shown that overexpression of dopamine type D2 (DA D2) receptors in the nucleus accumbens results in a marked reduction in alcohol intake in animals trained to self-administer alcohol. This effect returned to baseline levels as the DA D2 receptor concentration decreased to prior levels. Many clinical studies have noted the role played by dopamine and its receptors in alcohol, nicotine, and drug abuse. Animal studies indicate that DA D2 receptor levels mediate reinforcing responses to drugs of abuse. Human studies investigating the effects of DA D2 receptor antagonists on the reinforcing effects of psychostimulants have shown a decrease in the subjective ratings of pleasant sensations and of the craving associated with cocaine. Further support of these contentions includes evidence from gene polymorphisms. Research in animals over the past decade has established the central role of DA in cue-induced craving for addictive substances such as nicotine. In humans, smokers carrying either the DA D2 receptor gene TaqI A1 RFLP or the SLC6A3 (dopamine transporter gene) 9-repeat VNTR polymorphisms had stronger cue-induced cravings than non-carriers. Carriers of both polymorphisms had markedly higher craving responses in comparison to those who had neither. These findings support the role of DA in cue-induced craving in humans.

Much obesity research centers upon how the body's fuel and fat levels control appetite. However, habits and desires often override metabolic need. For example, recent obesity research indicates that too few hours per night of sleep causes under-production of certain hormones, specifically, too few of the hormones that control appetite. DA also appears to have a site-specific action in connection with food intake. Neuropeptide Y (NPY) is a potent stimulator of food intake. DA is an inhibitor of hypothalamic NPY expression and activity. DA also regulates food intake via the meso-limbic circuitry of the brain by apparently modulating appetitive motivational processes. Although dopaminergic effects in the nucleus accumbens are traditionally implicated in the motivation for food, a study in DA-deficient knockout mice provides clear evidence of relevance for the dorsal striatum as well. Dopamine D2 (DA D2) receptor levels have been found to vary inversely with body mass index in overweight humans. There is a large amount of evidence to suggest that dopamine may be one of the neurotransmitters linking the genetic and environmental factors that contribute to obesity. Behavioral studies on rodents indicate that DA D2 receptor antagonists are able to enhance meal size, duration of feeding, and body weight. In clinical studies, patients treated with both typical and atypical anti-psychotics, which block DA D2 receptors, show significant weight gain. Dopaminergic agonists that increase brain dopamine concentration have anorexigenic actions.

Human studies have shown a higher prevalence of the TaqI allele for the DA D2 receptor in obese subjects. This allele has generally been associated with lower levels of DA D2 receptors. The association of the TaqI allele with reduced numbers of DA D2 receptors suggests that the obese individuals with the A1 allele may use food to increase DA stimulation to a more desirable level. The DA system has also been targeted for therapy of obesity, since DA agonists have anorexigenic effects, whereas drugs that block DA D2 receptors increase appetite and result in weight gain.

It has been hypothesized that compulsive disorders as diverse as drug abuse, gambling, and sex-addiction reflect a ‘Reward Deficiency Syndrome’. This is speculated to be due, in part, to a reduction in DA D2 receptors. The dopaminergic system, and in particular the DA D2 receptor, has been implicated in reward mechanisms. The net effect of neurotransmitter interaction in the meso-limbic brain induces ‘reward’—when DA is released from the neuron at the nucleus accumbens and interacts with a DA D2 receptor. Because of these actions, DA has become known as the ‘pleasure’ molecule. When DA interacts with its receptor, it results in feelings of well-being. A consensus of the literature suggests that when a person has a dysfunction in his/her brain reward cascade, the person requires a DA ‘fix’ to feel good. This trait leads to multiple drug-seeking behaviors, and explains why a compulsive individual often has several inter-related addictions, e.g., for alcohol and smoking.

Prior to this disclosure, the above prior art existed only in isolated fragments from a variety of sources. Despite the strong evidence that there is a metabolic connection to addictive behavior, no one has yet devised a nutraceutical, functional food, dietary supplement, nutritional supplement, medical food, botanical drug, or drug effective for supporting and promoting a healthy lifestyle free from addictive behavior. Since such behavior has huge economic and human consequences, such a composition would be highly desirable.

### SUMMARY OF THE INVENTION

The invention provides new therapeutic, preventive, and health maintenance strategies which focus upon both direct and indirect modulation of dopaminergic function and restoration of associated brain circuitry. This is consistent with the observations that alcohol, cocaine, heroin, marijuana, nicotine use, glucose-bingeing, pathological gambling, excessive video game use, and sex-addiction all cause activation and release of brain DA. These aberrant behaviors, each related to self-fulfillment of a hypodopaminergic trait, may all be combined under the rubric of a reward deficiency syndrome.

The invention provides a composition for supporting and promoting a healthy lifestyle free from addictive behavior or for preventing or treating addictive behavior, the composition comprising a Vitex agnus-castus (Chastetree berry or Chasteberry) extract in an effective amount for supporting and promoting a healthy lifestyle free from addictive behavior or for preventing or treating addictive behavior. Preferably, the composition further comprises huperzine in an effective amount for supporting and promoting a healthy lifestyle free from addictive behavior or for preventing or treating addictive behavior. Preferably, the composition further includes biotin, L-carnitine, chromium polynicotinate, aspartic acid, and garcinia cambogia. Preferably, the composition may also include an ingredient selected from the group consisting of: EPA (eicosapentanoic acid), medium chain triglycerides, green tea leaf extract containing EGCG (epigallocatechingallate), and 5-HTP (5-hydroxytryptophan).

In an alternative embodiment, the composition includes Vitex agnus-castus extract and huperzine and further includes an ingredient selected from the group consisting of DMAE (dimethylaminoethanol) and choline.

In another embodiment, the composition comprises Vitex agnus-castus extract and huperzine, and also includes vitamin B1, vitamin B2, vitamin B3, vitamin B6, ferrous sulfate, Magnesium, choline, DHA (docosahexanoic acid), and lipoic acid. Additionally, the composition may include taurine.

In another alternative embodiment, the composition of the invention includes Vitex agnus-castus and 5-HTP (5-hydroxytryptophan). Preferably, the composition also includes tyrosine.

In any of the above embodiments, the composition may be included in a delivery vehicle comprising a vehicle selected from the group consisting of an edible film, a breath-care strip, mint or lozenge, a food, a beverage, a spice, a condiment, and a salad dressing.

The compositions above preferably also include an ingredient selected from the group consisting of glutamate agonists, antagonists, and precursors, GABA agonists, antagonists, and precursors, serotonin agonists, antagonists, and precursors, and agents that affect the levels and interactions of the forgoing ingredients, the ingredient provided in an effective amount for supporting and promoting a healthy lifestyle free from addictive behavior or for preventing or treating addictive behavior. The compositions above preferably also include ingredients to address nutritional deficiencies due to the addictive behavior, or ingredients to replenish or restore nutrients depleted by drugs taken for the addictive behavior. The compositions above preferably further include ingredients to replenish nutrients depleted during the withdrawal of any reinforcer that is the object of the addictive behavior.

The invention also provides a method for supporting and promoting a healthy lifestyle free from addictive behavior or for preventing or treating addictive behavior in a human being, the method comprising orally or parenterally administering to the human being, for an effective period, a composition comprising a Vitex agnus-castus extract in an effective amount for supporting and promoting a healthy lifestyle free from addictive behavior or for preventing or treating addictive behavior in a human being. Preferably, the method further includes administering an ingredient selected from the group consisting of huperzine and 5-HTP (5-hydroxytryptophan), the ingredient provided in an effective amount for supporting and promoting a healthy lifestyle free from addictive behavior or for preventing or treating addictive behavior. Preferably, the method further includes administering an ingredient selected from the group consisting of glutamate agonists, antagonists, and precursors, GABA agonists, antagonists, and precursors, serotonin agonists, antagonists, and precursors, and agents that affect the levels and interactions of the forgoing ingredients, the ingredient provided in an effective amount for supporting and promoting a healthy lifestyle free from addictive behavior or for preventing or treating addictive behavior. Preferably, the method further comprises administering an ingredient selected from the group consisting of: ingredients to address nutritional deficiencies due to the addictive behavior; ingredients to replenish or restore nutrients depleted by drugs taken for the addictive behavior; and ingredients to replenish nutrients depleted during the withdrawal of any reinforcer that is the object of the addictive behavior.

The invention not only provides a composition for supporting and promoting a healthy lifestyle free from addictive behavior or for preventing or treating addictive behavior in a human being; it also provides methods of delivering the composition that assist in its daily use. Numerous other advantages and features of the invention will become apparent from the following detailed description.

### DETAILED DESCRIPTION OF THE PREFERRED EMBODIMENT

1. Overview

The invention recognizes that the characteristics of compulsive overeating are consistent with at least some of the characteristics of drug-using behavior. Both compulsive overeating and drug addiction manifest the inability to refrain from using a reinforcer, and its compulsive administration. Thus, DA D2 decrements are unlikely to be specific for any one of these compulsive behavioral disorders, including obesity, and may relate to vulnerability for addictive disorders in general. Obese individuals have significantly lower DA D2 receptor levels, as do drug-addicted subjects. Lower DA D2 receptor levels in obese individuals would make them less sensitive to reward stimuli, which would make them more vulnerable to food intake as a means to temporarily compensate for this deficit.

Increases in DA secondary to phasic DA cell firing plays an important role in coding rewards and reward-associated stimuli, yet do not code exclusively for reward but also for saliency, which in addition to reward includes aversive, novel, and unexpected stimuli. It is proposed here that DA encodes for the motivation to procure the reward in addition to encoding for the reward itself. This view about the role of DA in reinforcement provides a different perspective about drugs of abuse, indicating that drugs are reinforcing not just because they are pleasurable but also because by increasing DA they are being processed as salient stimuli that will inherently motivate further drug procurement.

Just as compulsive behavior, such as drug-seeking, is encoded by DA (which attaches significance to such stimuli), hypodopaminergic conditions such as various attentional disorders may be unable to attach appropriate salience. It may be, then, that all stimuli are viewed as being equi-salient. This may make it impossible to prioritize, and may provide insight into the inability to focus or choose something specific to focus upon. From this perspective, it is not difficult to imagine a relationship between salience and inattention or impulsiveness. The converse may apply to the compulsive behavior of a drug addict wherein specific behavior (i.e., drug taking) is assigned salience, and is thereby reinforcing.

As has been described above, PET scans have shown that the reinforcing effects of drugs of abuse are contingent upon large and rapid increases in extra-cellular DA. In addition, similar studies have also documented a role for DA in motivation, which appears to be encoded by both fast, as well as less dramatic, DA increases. Since DA neurons fire in response to salient stimuli, supra-physiological activation by drugs is likely to be experienced as highly salient, in turn driving attention, arousal conditioned learning, and motivation. As noted above, imaging studies have shown in drug addicts that DA function is markedly disrupted, i.e., decreases in DA release and in DA D2 receptors occur in striatal regions, and this is associated with decreased activity of the orbito-frontal cortex (the neuroanatomical region involved with salience attribution and motivation, and implicated in compulsive behaviors) and the cingulate gyrus (the neuroanatomical region involved with inhibitory control and attention/focus/vigilance and which is implicated in impulsiveness). It is postulated here that decreased DA function results in decreased sensitivity to non drug-related stimuli and disruption of frontal inhibition. Such abnormalities have been described in persons afflicted with attention deficit disorder (ADD), attention deficit hyperactivity disorder (ADHD), and other conditions with altered attention processing. It is, therefore, no surprise that this population suffers from excessive addictive behavior and that appropriate medical therapy diminishes this risk.

The invention provides new therapeutic, preventive, and health maintenance strategies which focus upon both direct and indirect modulation of dopaminergic function and restoration of associated brain circuitry. This is consistent with the observations that alcohol, cocaine, heroin, marijuana, nicotine use, glucose-bingeing, pathological gambling, excessive video game use, and sex-addiction all cause activation and release of brain DA. These aberrant behaviors, each related to self-fulfillment of a hypodopaminergic trait, may all be combined under the rubric of a reward deficiency syndrome. Extracts from the Chastetree berry (Vitex agnus-castus) are germane in this context. They contain a number of compounds or constituents with dopaminergic properties. Specifically, they bind to recombinant DA D2 receptor protein with subsequent receptor activation. The chemical identity of the active compounds involves the isolation of a number of diterpenes. Hence, extracts of the Chastetree berry (Vitex agnus-castus) manifest dopaminergic activity and are specifically active at the DA D2 receptor. Herein, the term Vitex agnus-castus extract means any active compound derived from the Vitex agnus-castus plant, which is also referred to as Chastetree, including any compound derived from the Chastetree berry, which may also be referred to as the Chasteberry, and also including any drug or drug ingredient synthesized from the Vitex agnus-castus plant. Based upon the prior discussion, along with the ability of extracts of Vitex agnus-castus to cross the blood-brain barrier, clinical applications in a number of addictive conditions including, but not limited to, overeating, overweight, obesity (and related conditions), sugar and chocolate craving, smoking, excessive alcohol consumption, nicotine addiction, narcotic addiction, cocaine, and any number of other addictive drugs, compulsive gambling, and pathological sex-addiction exist. These agents directly augment DA D2 neurotransmission. In so doing, they provide the DA ‘fix’ the body would otherwise be seeking. This mechanism acts to circumvent the reward deficiency and forms the basis for the current therapeutic approach. They will also be effective in conditions manifesting impulsiveness, inattention, difficulty assigning salience, and the like. These extracts may also be combined with additional agents and ingredients having benefit in specific situations; for example, ingredients designed to replenish and restore nutrients or nutritional deficiencies in such addictive individuals, and nutrients depleted during the withdrawal of any drug, nicotine, or other reinforcer that is the object of the addiction.

The compositions according to the invention may be included in foods, “functional foods,” dietary supplements, medical foods, botanical drugs, homeopathic remedies, over-the-counter drugs, prescription drugs, and compounded drugs. They may be useful not only for the treatment of various addictions, but also in the prevention of such behaviors. These extracts and combinations may also be used as appetite suppressants for merely overweight (not obese) individuals, and to restore nutrients depleted in addictive-prone and compulsive individuals, and in those in withdrawal.

Formulations

Each formulation is designed to be taken by human beings, preferably on a daily basis, preferably several times per day for at least two weeks, and then thereafter for as long as is required. Each formulation includes Vitex agnus-castus, preferably as an extract, administered in capsules, soft gel tablets, liquids, etc., and may include other herbal, botanical, or dietary ingredients of the sort used in and permitted in dietary supplements, and any drug or drug ingredient synthesized from Vitex agnus-castus. Administration via the oral, sublingual, subcutaneous, intra-muscular, transdermal, or IV route is anticipated. Inclusion in foods, drinks, wafers, chewable formulations, gum, candy, bars, powdered shakes, meal replacements, dietary supplements, etc., are anticipated, as well as inclusion in functional foods, medical foods, drugs, etc. The compositions according to the invention may be included with many other combinations of compounds which are beneficial in the conditions listed, including, but not limited to vitamins, minerals, amino acids, and other nutrients and dietary ingredients. They may be included with many other combinations of compounds which are beneficial in the conditions listed for use in compounded drugs, prepared by a compounding pharmacist for a specific individual. One skilled in the field, such as a physician, may use medical insight, therapeutic skill, prior knowledge, data from the literature, and so forth to compose the specific formulation for a specific individual.

There is extensive interaction among numerous neurotransmitters in many ways. For example, acetylcholine may modulate dopamine neurotransmission. Therefore, agents that modify cholinergic neurotransmission such as huperzine may be expected to indirectly impact dopaminergic pathways and thereby influence the net effect that administration of Vitex agnus-castus has. This is also true for glutamate agonists, antagonists, and precursors, GABA agonists, antagonists, and precursors, serotonin agonists, antagonists, and precursors, and agents that affect their levels and interactions, and so forth, for most of the known neurotransmitters. One skilled in the art will have the benefit of the medical literature and past training for precise guidance in composing specific formulations based upon these principles.

### EXAMPLE 1

Overweight is a condition associated with the ingestion of a relative excess of calories. Frequently, under such circumstances, food is consumed for reasons other than metabolic need. Under these conditions, it may play a rewarding role and be associated with DA release. The provision of an extract of Vitex agnus-castus alone, or in combination with other agents, may substitute for the foodstuffs as a reinforcer and would act to diminish the reliance upon food, thereby facilitating subsequent weight loss.

Formulation 1

Vitex agnus-castus capsules: (standardized to casticin)—40 mg taken orally twice per day

Formulation 2

As above, but including the following per dose or serving size:

| | | |
|---|---|---|
| Biotin | 600 | mcg |
| L-carnitine | 10 | mg |
| Chromium polynicotinate | 400 | mcg |
| Aspartic acid | 4 | g |
| Garcinia cambogia | 500 | mg |

Formulation 3

As in Formulation 2 above, but including the following per dose or serving size:

| | |
|---|---|
| EPA (eicosapentanoic acid) | 10 mg per dose or serving size |

Formulation 4

As in Formulations 2 or 3 above, but including Medium Chain Triglycerides—5 g per dose or serving size

Formulation 5

As in Formulations 2, 3, or 4 above, but including the following per dose or serving size:

Green tea leaf extract containing 100 mg of EGCG (epigallocatechingallate)

Formulation 6

As in Formulations 2, 3, 4, or 5 above, but including the following per dose or serving size:

| | |
|---|---|
| 5-HTP (5-hydroxytryptophan) | 250 mg |

### EXAMPLE 2

For smoking cessation, one dose or serving size two or three times per day.

Formulation 1

Each dose or serving size consisting of Vitex agnus-castus (standardized to casticin)—50 mg

Formulation 2

As in Formulation 1 above, but including per dose or serving size:

| | |
|---|---|
| Huperzine | 50 mcg |

Formulation 3

As in Formulation 2 above, but including per dose or serving size:

| | |
|---|---|
| DMAE (dimethylaminoethanol) | 150 mg |

Formulation 4

As in Formulations 2 or 3 above, but including per dose or serving size:

| | |
|---|---|
| Choline | 200 mg |

### EXAMPLE 3

For use in conditions of attentional deficit, impulsiveness, or hyperactivity.

Formulation 1

Vitex agnus-castus (standardized to casticin)—100 mg per dose or serving size to be taken two or three times per day.

Formulation 2

As in Formulation 1 above, but including per dose or serving size:

| | |
|---|---|
| Huperzine | 100 mcg |

Formulation 3

As in Formulation 2 above, but including per dose or serving size:

| | | |
|---|---|---|
| Vitamin B1 | 10 | mg |
| Vitamin B2 | 20 | mg |
| Vitamin B3 | 25 | mg |
| Vitamin B6 | 10 | mg |
| Ferrous sulfate | 5 | mg |
| Magnesium | 150 | mg |
| Choline | 50 | mg |
| DHA (docosahexanoic acid) | 40 | mg |
| Lipoic acid | 30 | mg |

Formulation 4

As in Formulations 2 or 3 above, but also including per dose or serving size:

| | |
|---|---|
| Taurine | 50 mg |

### EXAMPLE 4

To help counteract excessive sweet cravings and as an appetite suppressant generally, taken several times per day and as needed, per dose or serving size:

Formulation 1

Extract of Vitex agnus-castus (standardized to casticin)—40 mg

Formulation 2

As in Formulation 1 above, plus per dose or serving size:

| | |
|---|---|
| 5-HTP (5-hydroxytryptophan) | 250 mg |

Formulation 3

As in Formulation 2 above, plus the following per dose or serving size:

| | |
|---|---|
| Tyrosine | 500 mg |

As discussed above, the above compositions according to the invention are preferably administered via the oral, sublingual, subcutaneous, intramuscular, transdermal, or IV route. Sometimes, such “formal” administration may not be effective, in that taking a dose of a “medicine” each day is difficult enough for healthy people, much less addicts. Therefore, the invention contemplates Inclusion of any of the above compositions in foods, drinks, wafers, chewable formulations, gum, candy, bars, powdered shakes, meal replacements, dietary supplements, etc., as well as inclusion in functional foods, medical foods, drugs, etc. For example, the delivery vehicle may comprise an edible film, a breath-care strip, mint or lozenge, or a food, water, beverage, spice or other food additive, condiment, or salad dressing or other functional food. The preferred foods are: energy bars, salad dressings, condiments (such as steak sauce, mustard, catsup, and soy sauce), vegetable oils, fruit products (such as jellies, jams and syrups), cereals, trail mix, cookies, pasta, flours (including wheat, soy, oat, and potato flour), whey, chocolate, yogurt, tofu, bagels, baked goods, vegetables, soups, nutritional bars, snacks, crackers, meats, and meat products such as lunch meats, and milk products such as ice cream, cheese, and butter. The term “beverage” is used in its common meaning, which does not include water or medicines. The preferred beverages are sports drinks, soft drinks, alcoholic beverages, tea, coffee, milk, and fruit juices.

There have been described novel compositions and methods for promoting compulsion-free health. It should be understood that the specific formulations and methods described herein are exemplary and should not be construed to limit the invention, which will be described in the claims below. Further, it is evident that those skilled in the art may now make numerous uses and modifications of the specific embodiments described without departing from the inventive concepts. For example, the appropriate assignment of salience, and/or importance, to various stimuli or tasks is also a part of non-compulsive behavior. The ability to attend and focus, especially for prolonged periods, also falls under the purview of this invention. Included are behaviors of compulsive and/or excessive food consumption, drug taking and/or addiction, smoking, compulsive, excessive or inappropriate gambling, compulsive or uncontrollable sexual behavior, chocolate or sweet craving, impulsive behavior, difficulty with concentration, attention and/or focus, and the like. Also contemplated are compositions and methods for dietary supplements to restore nutritive balance, and nutrients lost or depleted during any protocol to address or “break” an addiction. Consequently, the invention is to be construed as embracing each and every novel feature and novel combination of features present in and/or possessed by the compositions and methods described and by their equivalents.

1-14. (canceled)

15. A method for supporting and promoting a healthy lifestyle free from addictive behavior or for preventing or treating addictive behavior in a human being, said method comprising orally or parenterally administering to the human being, for an effective period, a composition comprising a Vitex agnus-castus extract in an effective amount for supporting and promoting a healthy lifestyle free from addictive behavior or for preventing or treating addictive behavior in a human being.

16. A method as in claim 15 wherein said method further includes administering an ingredient selected from the group consisting of huperzine and 5-HTP (5-hydroxytryptophan), said ingredient provided in an effective amount for supporting and promoting a healthy lifestyle free from addictive behavior or for preventing or treating addictive behavior.

17. A method as in claim 15 wherein said method further includes administering an ingredient selected from the group consisting of glutamate agonists, antagonists, and precursors, GABA agonists, antagonists, and precursors, serotonin agonists, antagonists, and precursors, and agents that affect the levels and interactions of the forgoing ingredients, said ingredient provided in an effective amount for supporting and promoting a healthy lifestyle free from addictive behavior or for preventing or treating addictive behavior.

18. A method as in claim 15 wherein said method further includes administering an ingredient selected from the group consisting of: ingredients to address nutritional deficiencies due to said addictive behavior; and ingredients to replenish nutrients depleted during the withdrawal of any reinforcer that is the object of said addictive behavior.

19. A method as in claim 16, said method further including administering biotin, L-carnitine, chromium polynicotinate, aspartic acid, and garcinia cambogia in an effective amount for supporting and promoting a healthy lifestyle free from addictive behavior or for preventing or treating addictive behavior in a human being.

20. A method as in claim 19, wherein said method further includes administering an ingredient selected from the group consisting of: EPA (eicosapentanoic acid), Medium Chain Triglycerides (MCTs), green tea leaf extract containing EGCG (epigallocatechingallate), and 5-HTP (5-hydroxytryptophan) in an effective amount for supporting and promoting a healthy lifestyle free from addictive behavior or for preventing or treating addictive behavior in a human being.

21. A method as in claim 16, wherein said method further includes administering an ingredient selected from the group consisting of DMAE (dimethylaminoethanol) and choline in an effective amount for supporting and promoting a healthy lifestyle free from addictive behavior or for preventing or treating addictive behavior in a human being.

22. A method as in claim 16, wherein said method further includes administering vitamin B1, vitamin B2, vitamin B3, vitamin B6, ferrous sulfate, Magnesium, choline, DHA (docosahexanoic acid), and lipoic acid in an effective amount for supporting and promoting a healthy lifestyle free from addictive behavior or for preventing or treating addictive behavior in a human being.

23. A method as in claim 16, said method further including administering taurine in an effective amount for supporting and promoting a healthy lifestyle free from addictive behavior or for preventing or treating addictive behavior in a human being.

24. A method as in claim 23, said method further including administering tyrosine in an effective amount for supporting and promoting a healthy lifestyle free from addictive behavior or for preventing or treating addictive behavior in a human being.

25. A method as in claim 15, said method further comprising administering said composition in a delivery vehicle selected from the group consisting of an edible film, a breath-care strip, mint or lozenge, a food, a beverage, a spice or other food additive, a condiment, and a salad dressing.
